(19) United States
(12) Patent Application Publication
(10) Pub. No.: US 2022/0062899 A1
(43) Pub. Date: Mar. 3, 2022

(54) Method, Apparatus and System to Detect Sub-Particle Flowrate in a Closed System

(71) Applicant: Mission Bio, Inc., South San Francisco, CA (US)

(72) Inventor: Adam Sciambi, South San Francisco, CA (US)

(21) Appl. No.: 17/423,194

(22) PCT Filed: Jan. 21, 2020
(86) PCT No.: PCT/US2020/014488

§ 371 Date: Jul. 15, 2021

Related U.S. Application Data
(60) Provisional application No. 62/794,379, filed on Jan. 18, 2019.

(51) Int. Cl. B01L 3/00; C12Q 1/6806; G01N 33/50; G01F 1/708

(52) U.S. Cl. CPC B01L 3/502715; C12Q 1/6806; G01N 33/5005; B01L 2300/0681; B01L 2400/0487; B01L 2200/16; B01L 2300/0867; G01F 1/7086

(57) ABSTRACT

The disclosure relates to method, apparatus and system to detect flowrate in a microfluidic system. An exemplary method to detect flowrate of a low-flow constituent in a fluidic circuit includes the steps of: (1) pneumatically driving a first fluid into the fluidic circuit, the first fluid including a first reagent, a first detection component and the constituent; (2) defining a sampling area in the fluidic circuit and exposing a microchannel in the sampling area to a wavelength configured to excite the first detection component to thereby provide a detection emission from the first detection component of the first reagent; (3) filtering the detection emission from the first reagent at an optical filer to substantially isolate a detection emission frequency; (4) determining flowrate of the constituent through the microchannel as a function of the isolated detection emission frequency. The flowrate of the constituent through the microchannel can be measured relevant to the flowrate of the first detection component through the microchannel. In certain embodiments, the first detection component comprises a fluorescent dye.

The instant application claims priority to the filing date of Provisional Application No. 62/794,379, filed Jan. 18, 2019; the specification of which is incorporated herein in its entirety.

### FIELD

The disclosure relates to method, apparatus and system to detect flowrate in a microfluidic system. In one embodiment, the disclosure relates to a method, apparatus and system to detect particle flowrate in a microfluidic system with fluidic channels in the range of about 0.1 micro-liter per minute (μL/min) to 10 milli-liter per minute (mL/min). The disclosed embodiments may be used to detect movement of components in biological samples such as tumor cells (e.g., circulating tumor cells) through a fluidic circuit.

### BACKGROUND

Biological samples from a subject often contain a large number of different components. For example, a sample of a subject's blood may contain free floating DNA and RNA, circulating cells, and many other components. The number and diversity of such components in a biological sample often complicates or prevents the accurate identification and/or quantification of specific components of interest within the sample, which would enable the diagnosis or monitoring of a condition in the subject, such as cancer.

For instance, circulating tumor cells (CTCs) are cells shed from tumors that enter into a subject's blood stream. Once in the blood, these cells can circulate through the subject's body, where they can invade other tissues and grow new tumors. CTCs are thus implicated in metastasis, which is the primary cause of death in subjects with cancer. Efforts to count CTCs have been hampered by the fact that CTCs are extremely difficult to detect. They are exceptionally rare, and may be difficult to distinguish from healthy cells. Current approaches for detecting CTCs rely on immunoassays, in which antibodies are used to target specific biomarkers on the surfaces of the CTCs. However, such approaches have limitations in sensitivity and/or specificity, leading to many healthy cells being mischaracterized as cancerous, and many cancer cells being missed in the analysis.

There is a need to identify and measure movement of biological components (e.g., particles) through a fluidic circuit used for cell testing and identification. Such measurement can inform other valuable information, for example, it can identify obstruction in a fluidic circuit. The fluidic flow in such system is exceptionally slow (low-flow rate) and the conventional systems do not provide a meaningful measure of particle movement in such systems that does not expose (i.e., contact-less) the particle.

### SUMMARY

Methods, system and apparatus for detecting component flowrate in microfluidic circuits are disclosed. In certain aspects, the methods may be used to detect and/or quantify specific component (e.g., particles) flowrate in a biological sample in a closed microfluidic system. The component may comprise tumor cells (e.g., circulating tumor cells, or CTCs), chemicals, droplets, particulate entities, molecules and the like. Systems and devices for use in practicing methods of the invention are also provided.

In one embodiment, the disclosure relates to method, apparatus and system to detect flowrate in a microfluidic system. An exemplary method to detect flowrate of a low-flow constituent in a fluidic circuit includes the steps of: (1) pneumatically driving a first fluid into the fluidic circuit, the first fluid including a first reagent, a first detection component and the constituent; (2) defining a sampling area in the fluidic circuit and exposing a microchannel in the sampling area to a wavelength configured to excite the first detection component to thereby provide a detection emission from the first detection component of the first reagent; (3) filtering the detection emission from the first reagent at an optical filer to substantially isolate a detection emission frequency; (4) determining flowrate of the constituent through the microchannel as a function of the isolated detection emission frequency. The flowrate of the constituent through the microchannel can be measured relevant to the flowrate of the first detection component through the microchannel. In certain embodiments, the first detection component comprises a fluorescent dye.

In another embodiment, the disclosure relates to a system to detect flowrate of a low-flow constituent in a fluidic circuit. An exemplary system comprises: a cartridge having one or more fluidic reservoirs and a sampling area wherein: the one or more one or more fluidic reservoirs are configured to receive a first fluid, the first fluid including a first reagent, a first detection component and the constituent; the sampling area positioned relative to the one or more fluidic reservoirs and having a microchannel, the microchannel exposable to an incoming excitation radiation and emitting at least one excitation signal when one of the first detection component is excited; a power source to pneumatically drive the first fluid from the one or more fluidic reservoirs to the microchannel; an illuminate source to illuminate the sampling area with a wavelength configured to excite the first detection component to thereby provide a detection emission from the first detection component; an optical filter to filter the detection emission from to substantially isolate a detection emission frequency; and a processor to receive the substantially isolated detection emission frequency and to determine flowrate of the constituent through the microchannel as a function of the isolated detection emission frequency. The flowrate of the constituent through the microchannel can be measured relevant to the flowrate of the first detection component through the microchannel. The detection component may include one or more fluorescent dyes or other chemicals that can be excited to emit a known-signature radiation.

In some embodiment, the flowrate of the low-flow constituent is in the range of about 0.1 μL/min to about 1 mL/min. In some embodiments, the flowrate of the low-flow constituent is equal or less than 1 μL/min. In one embodiment, the flow rate is about 0.1 μL/min.

An exemplary system according to one embodiment is contact-less. That is, a pneumatic power source drives the fluid into the reservoirs or from the reservoirs into one or more microchannels. Further, an illumination source illuminates a detection component (e.g., a fluorescent dye). Emissions from the illuminated detection component are received from the sampling area and used to track movement of the dye through the sampling area.

In certain embodiments, flow rates of two detection components are measured simultaneously and two flowrates are determined relative to each other. In one implementation a microprocessor circuitry measures flowrate of the second detection component through one or more microchannels to determine a relative movement of the first and the second detection components through the microchannel.

The detection system may include a memory circuitry to store information or instructions. The instructions may be executed on a microprocessor circuitry. The microprocessor circuitry may be in communication with the pneumatic driving mechanism to control fluidic flow through the microchannel. The microprocessor may also be in communication with the illumination source used to activate the detection component. The microprocessor may be in communication with an optical filter system that received radiation emissions from the detection component once the detection component is optically activated by the illumination source. The microprocessor can identify movement or placement of the detection component in the microchannel during a period of time to thereby calculate movement of the low-flow constituent in the microchannel. Thus, an exemplary system can measure flowrate of a low-flow constituent in a microchannel without contacting the constituent or its fluidic carrier.

In some embodiments, the flowrate of the constituent is used to determine movement of a discrete particle, an entity, a cell or a droplet through one or more microchannels.

In still another embodiment, the determined flowrate is compared to a threshold value to identify an obstructed microchannel. In another embodiment, the determined flowrate is compared to a threshold value to identify internal pressure in the microchannel.

### BRIEF DESCRIPTION OF THE DRAWINGS

The invention may be best understood from the following detailed description when read in conjunction with the accompanying drawings in which like elements are numbered similarly and where:

FIG. 1 is a flow diagram of a conventional genotyping technique;

FIG. 2 schematically illustrates an exemplary conventional cartridge to be used with an embodiment of the disclosure;

FIG. 3 is a first sideview of the exemplary cartridge of FIG. 2;

FIG. 4 is a second sideview of the exemplary cartridge of FIG. 2;

FIG. 5 is a photograph showing an implementation of an embodiment of the disclosure;

FIG. 6 is a block diagram of flow measurement system according to an exemplary embodiment of the disclosure; and

FIG. 7 is a flow diagram of an exemplary method according to an embodiment of the disclosure.

### DETAILED DESCRIPTION

Methods for the detection of components from biological samples are provided. In certain aspects, the methods may be used to detect and/or quantify specific components in a biological sample, such as tumor cells (e.g., circulating tumor cells). Systems and devices for use in practicing methods of the invention are also provided.

The subject methods and devices may find use in a wide variety of applications, such as the detection of cancer, detection of aneuploidy from DNA circulating in a mother's blood stream, monitoring disease progression, analyzing the DNA or RNA content of cells, and a variety of other applications in which it is desired to detect and/or quantify specific components in a biological sample.

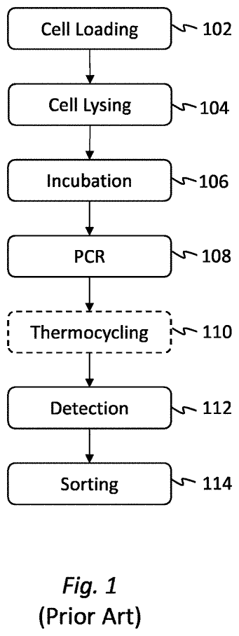

FIG. 1 is a flow diagram of a conventional genotyping technique. The flow diagram of FIG. 1 has been used to detect and/or genotype cells (e.g., tumor cells) from a biological sample. In one such method, nucleated blood cells are obtained from a biological sample. The nucleated blood cells are then encapsulated (loaded) into individual drops (step 102). Cell loading can be done with an encapsulation device. The drops are then injected with a cell lysing buffer (step 104) and incubated at conditions that accelerate cell lysis (step 106). The drops are then injected with a PCR mix that includes one or more primers targeting characteristic oncogenic mutations (step 108). Thermocycling (step 110) may be implemented optionally to activate PCR.

During the PCR reaction, if a droplet contains a genome of a cell with a mutation for which the primer(s) are designed to detect, amplification is initiated (step 110). The presence of a particular PCR product(s) may be detected by, for example, a fluorescent output that turns the drop fluorescent (step 112). The drops may thus be scanned (e.g., using flow cytometry) to detect the presence of fluorescently-tagged drops. The drops may also be sorted (step 114) using, for example, droplet sorting to recover drops of interest. The steps described above are conventionally performed under microfluidic control and with one or more microfluidics devices.

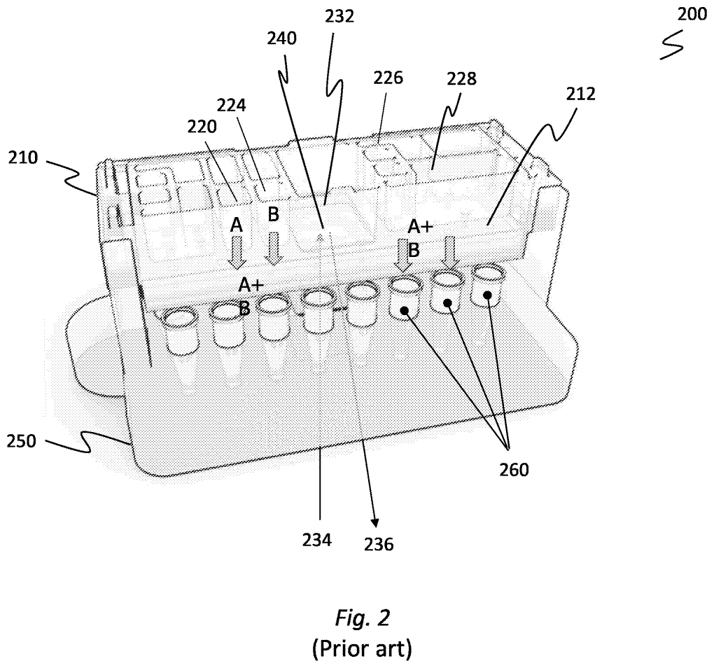

FIG. 2 schematically illustrates conventional microfluidics system 200 which can be used to detect fluid flow according to the disclosed embodiments. Microfluidic system 200 includes cartridge 210 and cartridge holder 250. Cartridge 210 may be formed as a thermoplastic part using injection molding or other similar methods. Cartridge 210 is shown with a series of reservoirs. For brevity, only fluid reservoirs 220, 224, 226 and 228 are shown. It is noted that the number of reservoirs and the reagents are exemplary in nature and not limiting of the disclosed principles. Each fluid reservoir is configured to receive one or more reagent. By way of example, FIG. 2 shows reservoir 220 receiving reagent A, reservoir 224 receiving reagent B and reservoir 228 receiving a combination of reagents A and B. The reagents may include detection components (e.g., fluorescent dyes or other conventional tags) which emit radiation when photoactivated.

The fluidic reservoirs are configured to receive reagents which can be used for automated testing, for example, as described in steps 102-106 of FIG. 1. External pressure (not shown) is used to drive fluids from the reservoirs through the microfluidic system 200. A series of PCR collection tubes 260 are positioned in cartridge holder 250. The PCR collection tubes 260 are positioned below reservoirs of cartridge 210. A plurality of reservoir openings 212 are formed at the bottom of cartridge 210 and are configured to communicate fluid out of each reservoir to each of the respective collection tubes 260. Fluid from cartridge 210 may be communicated to collection tubes 260, for example, by pneumatic pressurization of cartridge 210.

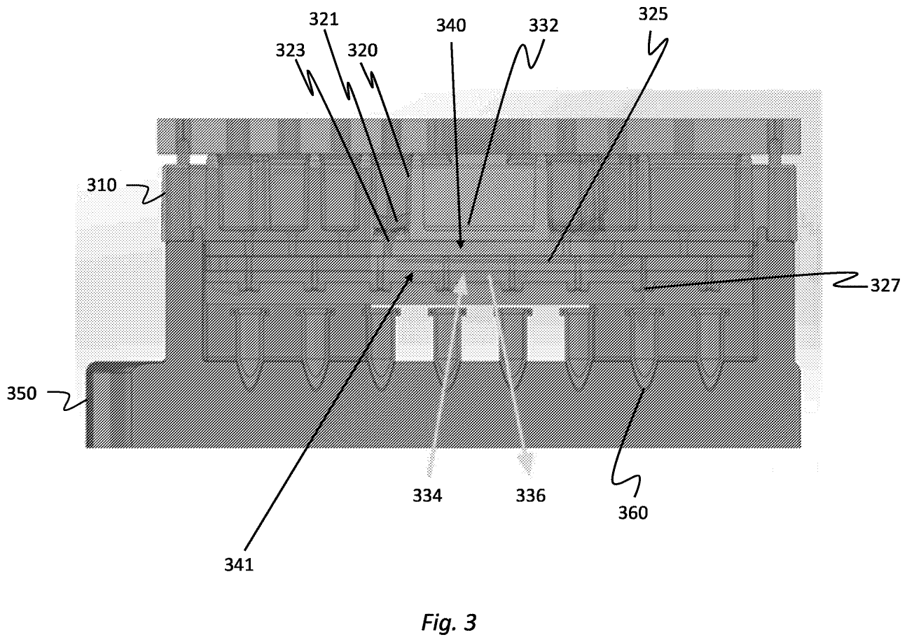

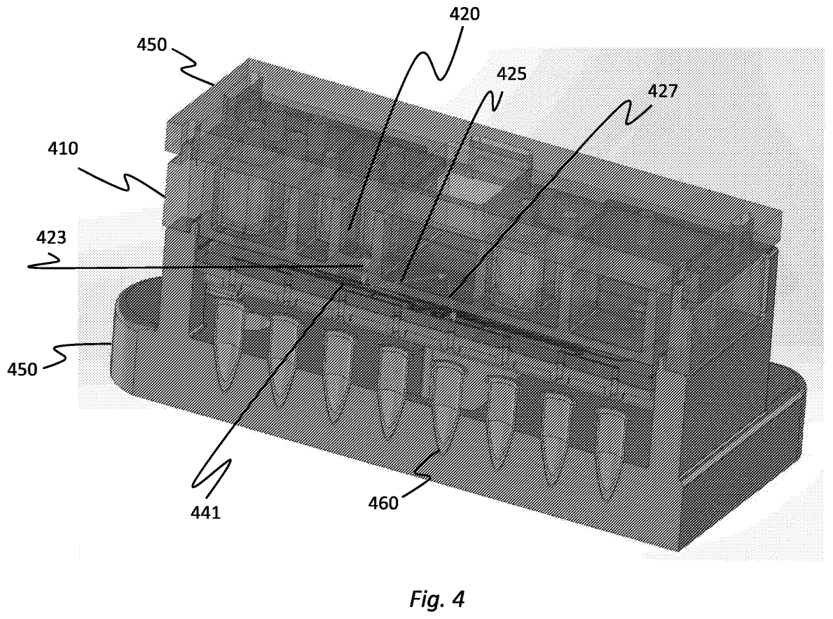

A flow detection region (interchangeably, sampling area) 232 is formed in cartridge 232. The location of detection region 232 in FIG. 2 is exemplary. One or more microchannels 240 are formed at flow detection region 232. Microchannels 240 are configured to receive one or more reagents, and/or mixtures thereof, from the fluid reservoirs (further illustrated in FIGS. 3 and 4). In one embodiment, microchannel 240 has inside diameter in the millimeter range. In one embodiment, microchannel 240 has inside diameter in the millimeter range. In still another embodiment, the microchannel has inside diameter in the micrometer range. In still another embodiment, the microchannel has inside diameter in the nanometer range.

Sampling area 232 may receive fluorescent excitation as indicated by arrow 234. Conventional fluorescent excitation source may be used for this purpose. Reagents having fluorescent tags will emit fluorescent light upon receiving excitation rays 234. A detector (not shown) receives fluorescent emission 236 and can measure reagent movement in microchannel 240 of flow detection region 232.

Various ways of detecting the absence or presence of PCR products has been conventionally employed in which a variety of different detection components are used. Detection components of interest may include, among others, fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Detection components may include beads (e.g., magnetic or fluorescent beads, such as Luminex beads) and the like. Detection may involve holding a microdroplet at a fixed position during thermal cycling so it can be repeatedly imaged. In certain aspects, detection may involve fixing and/or permeabilizing one or more cells in one or more microdroplets.

Referring again to FIG. 2, one or more detection components is excited 234 by an illumination source (not shown) at sampling area 232. Once excited, the detection components emit excitation rays 236. The emission is be detected via one or more detection systems which are further described below. An exemplary detection system may include a photodiode or charge-coupled device (CCD) to detect fluorescence emission signals.

FIG. 3 is a first sideview of the exemplary cartridge of FIG. 2. Specifically, FIG. 3 shows cartridge 310 positioned inside cartridge holder 350. Cartridge holder 350 includes a plurality of collection tubes 360 and a plurality of reservoirs. For brevity, only reservoir 320 is numbered. Reservoir 320 is shown with reagent 321.

Cartridge 310 includes flow detection area (sampling area) 332 which includes one or more microfluidic channels (not shown). Flow detection region 332 is configured to receive fluorescence excitation rays as indicated by arrow 334. Fluorescence emission rays 336 are generated from detection component(s) included in the reagent flowing through the microchannel 341.

The reagent flow through cartridge 310 is illustrated by arrows 323, 325 and 327. In one implementation, pneumatic pressure causes movement of reagent 321 through reservoir 320 and into the microchannel 341 as indicated by arrows 323 and 325. In one embodiment of the disclosure, the reagent flow through microchannel 341 is equal or less than 1 μL/min. In another embodiment, the reagent flow through microchannel 341 is equal or less than 1 μL/min. During reagent flow through microchannel 340, an excitation source illuminates 334 detection components. The detection components emit excitation rays 236. The emission is detected via one or more detection systems (not shown). The effluent of microchannel 340 is collected at collection tube 360 as indicated by arrow 327.

FIG. 4 is a second sideview of the exemplary cartridge of FIG. 2. In FIG. 4, cartridge 410 is placed inside cartridge holder 450. Reservoir 420 contain an exemplary reagent which is pressure fed (e.g., pneumatically) through one or more microchannels 441 to collection tube 460 as indicated by the plurality of arrows 423, 425 and 427.

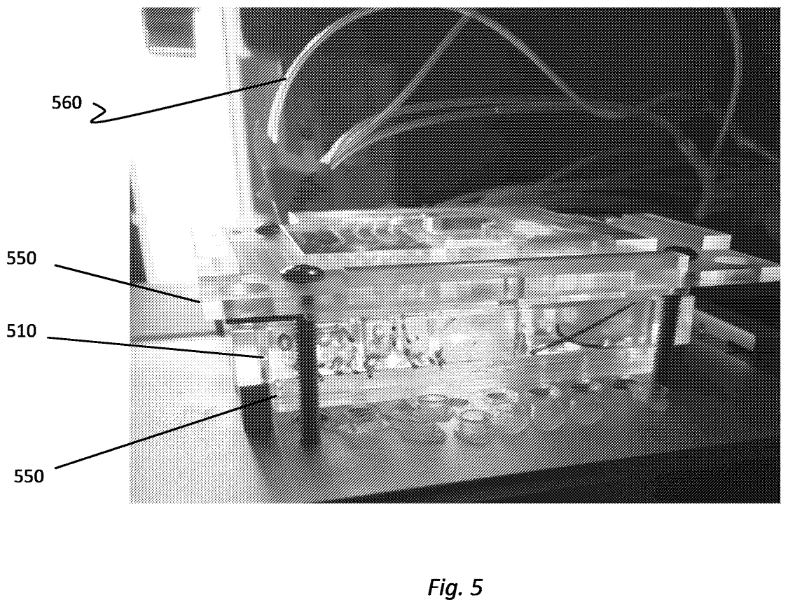

FIG. 5 is a photograph showing an implementation of an embodiment of the disclosure. Specifically, FIG. 5 is a photograph of real-time flow measurement using fluorescent illumination. In FIG. 5 cartridge 510 is placed inside cartridge holder 550. Tubes 560 deliver reagent and pneumatic pressure to cartridge 510. In FIG. 5, the reagents are illuminated with fluorescent lights of different frequencies. It can be seen from FIG. 5 that different detection components identifying presence of different reagents (in real-time) are illuminated as indicated by the different colors emitted from the cartridge's detection region 510.

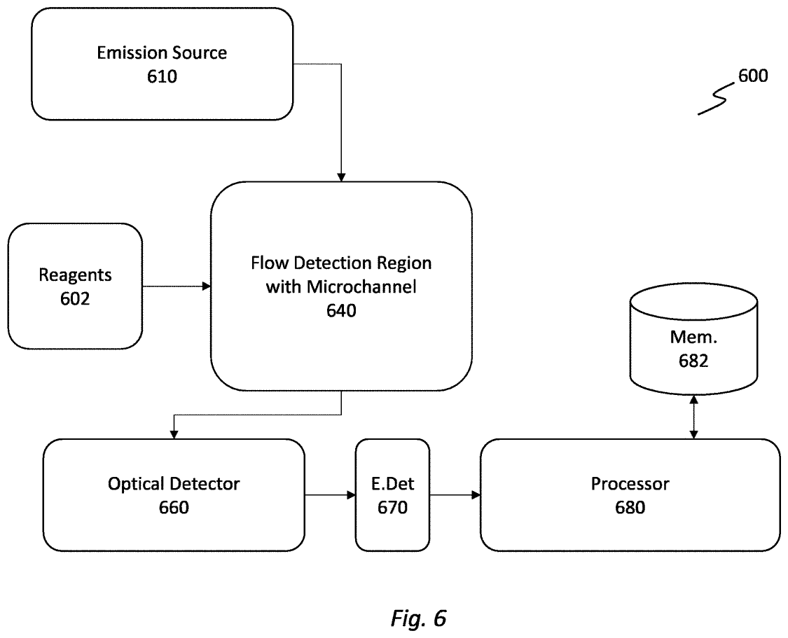

FIG. 6 is a block diagram of flow measurement system according to an exemplary embodiment of the disclosure. The flowrate measurements of system 600 can be made in real-time. In FIG. 6, System 600 includes reagent input 602, flow detection region (sampling area) 640, emission source 610, optical detector 660, electronic detector 670, processor circuitry 680 and memory circuitry 682. Reagent input 602 may comprise tubing, valves and pressure source necessary to move reagents into flow detection region 640 of a cartridge. Flow detection region 640 may include one or more microchannels to receive reagents 602. Reagents 602 may include one or more biological samples. The biological samples from a subject may contain a large number of different components. Components of interest include, but are not necessarily limited to, cells (e.g., circulating cells and/or circulating tumor cells), polynucleotides (e.g., DNA and/or RNA), polypeptides (e.g., peptides and/or proteins), and many other components that may be present in a biological sample. The subjects may be mammals or mammalian. The terms mammal and mammalian are used broadly to describe organisms which are within the class Mammalia. The disclosed embodiments are suitable for, among others, subjects in need of assessment according to the present disclosure.

Suitable subjects include those who have and those who have not been diagnosed with a condition, such as cancer. Suitable subjects include those that are and are not displaying clinical presentations of one or more cancers. In certain aspects, a subject may one that may be at risk of developing cancer, due to one or more factors such as family history, chemical and/or environmental exposure, genetic mutation(s) (e.g., BRCA1 and/or BRCA2 mutation), hormones, infectious agents, radiation exposure, lifestyle (e.g., diet and/or smoking), presence of one or more other disease conditions, and the like. A variety of different types of biological samples may be obtained from such subjects. In certain embodiments, whole blood is extracted from a subject. Whole blood may be treated prior to practicing the subject methods, such as by centrifugation, fractionation, purification, and the like. The volume of the whole blood sample that is extracted from a subject may be 100 mL or less, e.g., about 100 mL or less, about 50 mL or less, about 30 mL or less, about 15 mL or less, about 10 mL or less, about 5 mL or less, or about 1 mL or less.

The subject methods and devices provided herein are compatible with both fixed and live cells. In certain embodiments, the subject methods and devices are practiced with live cells. In other embodiments, the subject methods and devices are practiced with fixed cells. Fixing a cellular sample allows for the sample to be washed to extract small molecules and lipids that may interfere with downstream analysis. Further, fixing and permeabilizing cells allows the cells to be stained with antibodies for surface proteins as well as intracellular proteins. Combined with the Reverse-Transcriptase polymerase chain reaction (RT-PCR) methods described herein, such staining can be used to achieve high levels of multiplexing because the antibodies are localized to the cell sample, while RT-PCR products are free within a microdroplet. Such a configuration allows for dyes of the same color to be used for antibodies and for amplicons produced by RT-PCR. Any suitable method can be used to fix cells, including but not limited to, fixing using formaldehyde, methanol and/or acetone. The cell may be coupled to an identifying tag. The tag may be optically (or chemically) activated to identify its presence and thereby denote presence (or absence) of a component of interest.

Referring again to FIG. 6, optical detector 660 may include an optical train having one or more lenses, optical and/or electronic filters. The optical detector is configured to receive optical emission from detection component and communicate the received optical components to the electronic detector 670. Electronic detector 670 may be a CCD or a photodiode or the like. Electronic detector 670 receives converts the optical signal received from optical detector 660 to an electronic signal and communicates the electronic signal to processor circuitry 680.

Processor circuitry 680 may comprise hardware, software or a combination of hardware and software (i.e., firmware). Processor circuitry 680 may comprise instructions to process signals received from electronic detector 680 and determine presence and movement of particles through microchannel 640. Once a detection component is identified through its emission frequency, its movement through the microchannel can be measured in relation to time to thereby provide an estimate of component's flowrate. To the extent that the detection component is associated with a cell, droplet or other particulate samples passing through the sampling area, the flowrate will be indicative of the sample through the microchannel.

Processor circuitry 680 may also comprise instructions that allows determination of non-movement (e.g., clogging) of the microchannel 640. Such instructions may be stored at memory circuitry 682 and executed on processor circuitry 680. In some embodiments, processor circuitry 640 may execute instructions to detect particle movement in microchannel 640 as slow as 1 μL/min or less. In another embodiment, the particle movement in microchannel 640 as slow as 1 μL/min or less. In still another embodiment, the particle movement may be at least 1 μL/min or higher. In yet another embodiment, detected particle movements of two or more particles may be measured by system 600. In an exemplary implementation, the movement may denote cell movement or migration across microchannel 640.

In an exemplary embodiment, processor circuitry 680 and memory circuitry 682 may comprise a comparator. The comparator can be configured to compare the detected flowrate with a threshold value to identify an obstruction in the microchannel. In another exemplary embodiment, the detected constituent flowrate is compared with a threshold value to identify internal pressure (or lack thereof) in the microchannel.

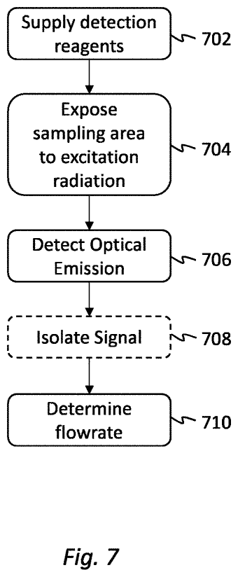

FIG. 7 is a flow diagram of an exemplary method according to an embodiment of the disclosure. The flow diagram of FIG. 7 starts at step 702 where reagents are provided into a sampling area. The supplied reagent may consist of one or more constituents coupled to (or associated with) a detection component. The reagent may be liquid, gel or one or mor solid particles combined with a liquid or a gel. The detection component may be activated chemically, electromagnetically or optically. In an exemplary embodiment, the detection component is a fluorescent device and is optically activated by exposure to appropriate fluorescent radiation. For example, the reagent and the associate detection component may be exposed to excitation radiation at the sampling area as shown in step 704. Once exposed (or activated), the detection component will emit an optical signal (e.g., fluorescent signal). At step 706, the emitted signal is detected with one or more optical and electronic component. At step 708, the detected emission signal may be isolated to reduce background noise from the signal. Step 708 is an optional step. At step 710, flowrate of the one or more constituent associated with the detection component is determined, for example, by tracking the movement of the detection component through the sampling area.

It will be apparent to those skilled in the technology of image displays that numerous changes and modifications can be made in the preferred embodiments of the invention described above without departing from scope of the invention. Accordingly, the foregoing description is to be construed in an illustrative and not in a limitative sense, the scope of the invention being defined solely by the appended claims.

What is claimed is:

1. A method to detect real-time flowrate of a low-flow constituent in a fluidic circuit, the method comprising:
pneumatically driving a first fluid into the fluidic circuit, the first fluid including a first reagent, a first detection component and the constituent;
defining a sampling area in the fluidic circuit and exposing a microchannel in the sampling area to a wavelength configured to excite the first detection component to thereby provide a detection emission from the first detection component of the first reagent;
filtering the detection emission from the first reagent at an optical filer to substantially isolate a detection emission frequency;
determining flowrate of the constituent through the microchannel as a function of the isolated detection emission frequency;
wherein the flowrate of the constituent through the microchannel is measured relevant to the flowrate of the first detection component through the microchannel and wherein the first detection component comprises a fluorescent dye.

2. The method of claim 1, wherein the flowrate of the low-flow constituent is in the range of about 0.1 μL/min to about 1 mL/min.

3. The method of claim 2, wherein the flowrate of the low-flow constituent is equal or less than 1 μL/min.

4. The method of claim 1, further comprising pneumatically driving a second fluid into the microchannel, the second fluid having a second reagent, a second detection component and a second constituent to substantially dilute presence of the first reagent in the sampling area.

5. The method of claim 4, further comprising measuring the flowrate of the second detection component through the microchannel to determine a relative movement of the first and the second detection components through the microchannel.

6. The method of claim 1, wherein the flowrate of the constituent is used to determine movement of a discrete particle, a cell or a droplet through the microchannel.

7. The method of claim 1, wherein the determined flowrate is compared to a threshold value to identify an obstructed microchannel.

8. The method of claim 1, wherein the determined flowrate is compared to a threshold value to identify internal pressure in the microchannel.

9. A system to detect flowrate of a low-flow constituent in a fluidic circuit, the system comprising:
a cartridge having one or more fluidic reservoirs and a sampling area wherein:
the one or more one or more fluidic reservoirs are configured to receive a first fluid, the first fluid including a first reagent, a first detection component and the constituent;
the sampling area positioned relative to the one or more fluidic reservoirs and having a microchannel, the microchannel exposable to an incoming excitation radiation and emitting at least one excitation signal when one of the first detection component is excited;
a power source to pneumatically drive the first fluid from the one or more fluidic reservoirs to the microchannel;
an illuminate source to illuminate the sampling area with a wavelength configured to excite the first detection component to thereby provide a detection emission from the first detection component;
an optical filter to filter the detection emission from to substantially isolate a detection emission frequency; and
a processor to receive the substantially isolated detection emission frequency and to determine flowrate of the constituent through the microchannel as a function of the isolated detection emission frequency;
wherein the flowrate of the constituent through the microchannel is measured relevant to the flowrate of the first detection component through the microchannel and wherein the detection component comprises a fluorescent dye.

10. The system of claim 9, wherein the flowrate of the low-flow constituent is in the range of about 0.1 μL/min to about 1 mL/min.

11. The system of claim 9, wherein the flowrate of the low-flow constituent is equal or less than 1 μL/min.

12. The system of claim 9, wherein the power source pneumatically drives a second fluid into the microchannel, the second fluid having a second reagent and a second constituent to substantially dilute presence of the first reagent in the sampling area.

13. The system of claim 12, wherein the microprocessor measures the flowrate of the second detection component through the microchannel to determine a relative movement of the first and the second detection components through the microchannel.

14. The system of claim 9, wherein the flowrate of the constituent is used to determine movement of a discrete particle, a cell or a droplet through the microchannel.

15. The system of claim 9, wherein the determined flowrate is compared to a threshold value to identify an obstructed microchannel.

16. The system of claim 9, wherein the determined flowrate is compared to a threshold value to identify internal pressure in the microchannel.
